(19) United States
(12) Patent Application Publication
(10) Pub. No.: US 2011/0218571 A1
(43) Pub. Date: Sep. 8, 2011

(54) ARTICULATED INTERVERTEBRAL SURGICAL IMPLANT TO ENCOURAGE CERTAIN INTERVERTEBRAL MOVEMENTS

(76) Inventor: David Attia, Montelimar (FR)

(21) Appl. No.: 12/597,152

(22) PCT Filed: Jun. 12, 2007
(86) PCT No.: PCT/FR2007/051424

§ 371 Date: May 11, 2011

(30) Foreign Application Priority Data
Apr. 24, 2007 (FR) 0754672

(51) Int. Cl. A61B 17/70

(52) U.S. Cl. 606/248

(57) ABSTRACT

The invention relates to a surgical implant intended to maintain and assist with the relative movement with respect to one another of two successive vertebras so as to obtain dynamic stabilization, said implant comprising an upper piece (2) and a lower piece (3) which are intended each to be associated with the processus spinosus, or spinal process, of one of said vertebras, said pieces each comprising a means (4, 5) of association with a spinal process and means of connection to the other piece.

According to the invention, said connecting elements are designed to collaborate in such a way as to form a pivot joint, at least one of said connecting elements being a visco-elastic element.

The invention relates to a surgical implant intended to maintain and assist with the relative movement with respect to one another of two successive vertebras in case of intervertebral instability, compressed disc or herniated disc.

The distension of disc and ligament structures gives rise to an excessive movement of one vertebra with respect to the other, mainly through a frontward and backward sliding which entails a shearing of nerve roots.

The most widely used treatment is arthrodesis which consists in merging the two vertebras together, but this entails a theoretical overloading of the underlying and overlying discs and the suppression of mobility of the operated area with a corresponding lost of flexibility.

The compression of a disc causes a shrinkage of the lateral holes through which root nerves are routed, which entails radicular pains or neurological disorder resulting from the compression of such nerve roots. The distension of disc and ligament structures causes the protrusion thereof into the vertebral channel, which also entails the shrinkage thereof.

Eventually, the existence of a voluminous lumbar disc hernia requiring a surgical removal may lead to foresee a compression of the disc after the operation.

In the three above situations, it is desirable to be able to position an interspinal implant making it possible to limit certain types of movement between two vertebras while allowing other movements. The aim is more particularly to provide an articulation enabling lateral inclination movements while limiting, without prohibiting them totally, the compression, traction, torsion, shearing and antero-posterior flexion movements.

In addition, it is advisable to maintain a gap between the spinal apophyses to correct a possible compression of the posterior part of the disc, to tighten the posterior ligamental structures, thus implying a widening of the channel and foramen.

The document FR 2884136 provides a surgical implant intended to maintain and assist with the relative movement with respect to one another of two successive vertebras enabling an efficient tightening of the implant about the spinal apophyses of the vertebras. This implant comprises an upper part and a lower part with a ball joint, each of said pieces includes means of association to a spinal apophysis of a respective vertebra.

This surgical implant also includes a visco-elastic element interposed between the upper and lower pieces at the ball joint, enabling the absorption of the energy of the shocks and a relative movement between the vertebras. This interposed intermediate element provided with some elasticity enables a deformation on compression and the distraction between the upper and lower parts. Thus, the visco-elastic element enables the flexion, extension or lateral inflexion of the vertebral column.

The ball joint formed by reciprocal association means of one piece and the other makes it possible to form an articulation assisting with the movement with respect to one another of the vertebras, the vertebral column thus having a natural mobility without excessive movement, a mobility which is identical to a sound rachis. The clearance between the means of association to the spinal apophyses and such apophyses can be eliminated to enable the movement with respect to one another of the vertebras, which gives a better holding of the implant between the vertebras.

On the contrary, such an implant does not limit certain types of movement which are tentatively avoided in the above mentioned cases. More particularly, it does not limit the antero-posterior mobility which is tentatively avoided.

The object of the present invention is more particularly to improve a surgical implant which has just being mentioned, thus enabling to solve the problem examined hereabove.

For this purpose, it provides a surgical implant comprising two articulated pieces, with each of said pieces including means of association to a spinal apophysis of respectively two successive vertebras, characterised in that said pieces include respective connecting elements adapted to collaborate in such a way as to form a pivot joint, with at least one of said connecting elements being a visco-elastic element.

The pivot joint between the two elements enables a pivoting movement with respect to one another of the vertebras in the direction of a lateral inclination. As one of the articulation elements is a visco-elastic element, other movements between the vertebras are enabled, although with a reduced amplitude.

Advantageously, said connecting elements further include means for limiting the displacement of said pivot joint.

Advantageously, said pivot joint allows the inclination by more or less 10° of one of said pieces with respect to the other.

Advantageously, said implant includes means for locking said connecting elements. Such means makes it possible to avoid the two pieces of the implant to be separated from each other, for example when traction is exerted when the connecting elements are flanged ball joint elements as described hereabove.

According to another aspect of the present invention, said connecting elements are two respectively male and female elements of a ball joint, clamping means making it possible to limit the movement between the pieces to a pivoting movement.

Advantageously, said male element of said ball joint is a visco-elastic element.

Advantageously, said clamping means include at least one rod connected to one of the pieces engaged in the two-lobed hole provided on the other piece.

Advantageously, said at least one two-lobed hole is provided on said male element of the ball joint.

Advantageously, said male and female elements of the ball joint have a spherical form.

Other particularities and advantages of the invention will appear when reading the following description and referring to the following figures, wherein:

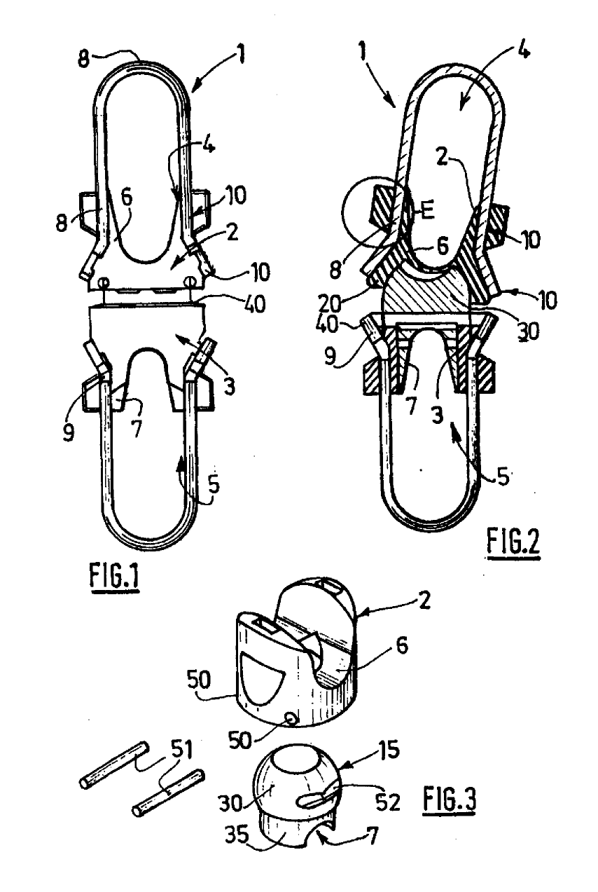

FIG. 1 is a schematic front representation of a surgical implant according to the invention.

FIG. 2 is a schematic sectional representation of the implant of FIG. 1.

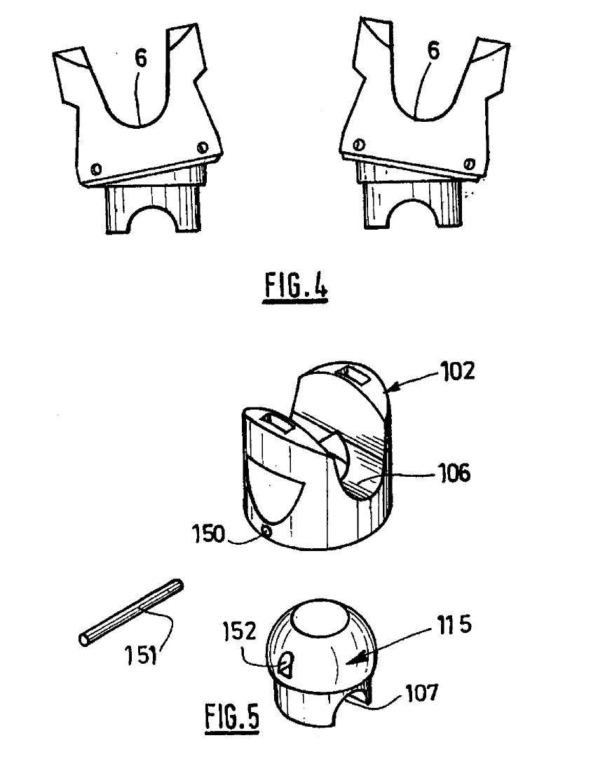

FIG. 3 is a schematic perspective representation of a part of the joint of the implant of FIG. 1.

FIG. 4 is a front schematic representation of the part of the joint of the implant of FIG. 1 in two extreme inclination positions.

FIG. 5 is a view similar to that of FIG. 3 of a part of the joint of the implant according to an alternative embodiment of the present invention.

While referring to FIGS. 1 and 2, a surgical implant 1 is described which comprises an upper piece 2 and a lower piece 3 intended to be associated, each to the spinal apophysis of two successive vertebras positioned one above the other.

As described in the above mentioned document FR 2884136, the upper piece 2 and lower piece 3 include means of association, respectively 4 and 5 with an apophysis. The means of association 4 and 5 each include a groove, respectively 6 and 7, and fixation means, respectively 8 and 9, intended for the fixing on the spinal apophysis of the vertebra which the piece is intended to be associated with.

The groove 6 of the upper piece 2 has a shape which substantially matches the lower part of the spinal apophysis of the vertebra which the piece 2 is intended to be associated with. As shown in FIG. 2, the groove 6 has a section which substantially has the shape of a U. As a matter of fact, the groove 6 is intended to receive the lower part of a spinal apophysis which is thicker than the upper part of this apophysis and so it is provided to adapt the shape of the groove 6 to that of the lower part of the apophysis in order to prevent a major intervention of the apophysis when positioning the implant. The U-shaped section makes such adaptation possible.

The groove 7 of the lower piece 3 has a shape which is substantially matching that of the upper part of the spinal apophysis of the vertebra which the piece 3 is intended to be associated with. As represented in FIG. 2, the groove 7 has a section which substantially has the shape of a V. As a matter of fact, the groove 7 is intended to receive the upper part of a spinal apophysis and as mentioned above, the latter is not as thick as the lower part of such apophysis. Then, it is planned to adapt the shape of the groove 7 to that of the upper part of the apophysis so as to prevent again major interventions on the apophysis when positioning the implant. The V-shaped section makes such adaptation possible.

The upper piece 2 and lower piece 3 are mainly made in a material making it possible to give the implant some rigidity. For example, a material such as PEEK is used since it has mechanical characteristics which are close to that of the bones. But it is also possible to make such pieces 2 and 3 from other plastic materials or metallic materials such as titanium or stainless steel.

According to the invention, the upper and lower pieces 2 and 3 each include respective connecting elements 20 and 30 adapted to collaborate in such a way as to form a pivot joint.

In the case of the upper piece 2, as can be best seen in FIG. 2, the hole 20 has a spherical shape and is provided in the lower part of the piece. This connecting element 20 is thus rigid, with the piece 2 being made in one piece from a rigid material such as PEEK.

The connecting element 30 of the lower piece 3, is a spherically shaped head 30, the dimensions of which are adapted to the hole 20 in such a way as to form a ball joint with the latter.

The connecting element 30 belongs to a sub-assembly 15 of the piece 3 made of a visco-elastic material. This sub-assembly 15 which is be visible in FIG. 3 includes a cylindrical lower part 35 adapted to be introduced into a hole provided for this purpose at the upper part of the piece 3, so that the sub-assembly 15 coaxially engages with the piece 3, the head 30 protruding upward, and the periphery of the lower end thereof which is plane, resting on the also plane upper surface 40 of the piece 3. The sub-assembly 15 is locked on the piece 3 for example using a pin (not shown) engaged through the ends thereof with holes being provided in the piece 3 and going through a hole (not shown) provided in the cylindrical lower part 35.

Thus, the spherical connecting head 30 of the lower piece 3, locked on the upper part of the latter is made of a visco-elastic material. Advantageously, it is a polymer material such as polyurethane (PU) or polycarbonate urethane (PCU).

In an alternative solution, the sub-assembly 15 can be over-moulded on piece 3. For example, the sub-assembly 15 made of PCU can be over-moulded onto the piece 3 made of PEEK.

According to the invention, said connecting elements 30 and 20 are two respectively male and female elements of a ball joint, clamping means 50, 51 and 52 making it possible to limit the movement between the pieces to a pivoting movement.

The clamping means are made of pins 51 adapted to be introduced into orifices 50 provided on either side of the upper piece 50, perpendicularly to the axis along which the groove 6 extends. Thus, these pins extend parallel inside the hole 20 of the upper piece 2.

Two-lobed holes of the “dolly” type are provided on either side of the head 30. These holes are adapted to receive each a pin 51. The spacing between the pins and the thickness of the holes is provided in such a way that the head 30 can pivot without deforming only in a plane parallel to said pins. Thus, the ball joint is clamped so as to form a pivot joint along an axis parallel to the axis of grooves 6 and 7. On the contrary, the deformation of said head enables other movements than this pivoting movement. Of course, the amplitude of these movements is much less than that of the pivoting movement. The “dolly” shape of the holes limits the displacement of the pieces with respect to each other. FIG. 4 shows the two extreme positions for the pivoting movement, respectively inclined by more or less 10° with respect to a straight position.

The pins further provide the locking of the ball head in the hole 20. Then, when an important traction is exerted, the pins ensure that the two pieces of the implant are not separated.

According to an alternative to this present solution, shown in FIG. 5, only one pin 151 goes through the female connecting element of an upper piece 102. This pin engages into a “dolly” hole, having a thickness corresponding to the diameter of the pin. Then again, the ball joint is flanged in such a way as to form a pivot joint, but the deformation of said head enables other movements than this pivoting movement. The “dolly” shape of the hole limits the displacement of the pieces with respect to each other. The pin engaged with the hole further ensures the locking of the ball head in the hole.

The fact that the ball head is a visco-elastic element allows the deformation of an implant, more particularly in compression and in traction, which gives it a characteristic of dynamic stabiliser with a damping effect and a traction resisting effect.

1. A surgical implant including two articulated pieces (2, 3), each of said pieces including means of association (4, 5) with a spinal process of two successive vertebras respectively, characterised in that said pieces include respective connecting elements (20, 30) adapted for collaborating in such a way as to form a pivot joint, one of said connecting elements (30) being a visco-elastic element.

2. An implant according to claim 1, characterised in that said connecting elements (20, 30) also include means (50, 51, 52; 150, 151, 152) for limiting the displacement of said pivot joint.

3. An implant according to claim 2, characterised in that said pivot joint allows an angle of inclination of more or less 10° of one of said pieces with respect to the other.

4. An implant according to any one of the preceding claims, characterised in that it further includes means (50, 51, 52; 150, 151, 152) for locking said connecting elements.

5. An implant according to any one of the preceding claims, characterised in that said connecting elements (30, 20) are two respectively male and female elements of a ball joint, clamping means (50, 51, 52; 150, 151, 152) making it possible to limit the movement between the pieces to a pivoting movement.

6. An implant according to claim 5, characterised in that said male element (30) of said ball joint is a visco-elastic element.

7. An implant according to claim 5 or 6, characterised in that said clamping means include at least one rod (51; 151) connected to one of the pieces (2) engaged in a two-lobed hole (52; 152) provided on the other piece (3).

8. An implant according to claim 7, characterised in that said at least one two-lobed hole (52; 152) is provided on said male element (30) of the ball joint.

9. An implant according to any one of claims 5 to 8, characterised in that said male (30) and female (20) elements of the ball joint have a spherical shape.
